(19) United States
(12) Patent Application Publication
(10) Pub. No.: US 2024/0117320 A1
(43) Pub. Date: Apr. 11, 2024

(54) ENGINEERED VASCULAR TISSUE MODELS

(71) Applicants: Duke University, Durham, NC (US); The Johns Hopkins University, Baltimore, MD (US)

(72) Inventors: Sharon Gerecht, Durham, NC (US); Danielle Yarbrough, Durham, NC (US); Franklyn Hall, Durham, NC (US)

(21) Appl. No.: 18/484,137

(22) Filed: Oct. 10, 2023

Related U.S. Application Data
(60) Provisional application No. 63/414,728, filed on Oct. 10, 2022.

(51) Int. Cl. C12N 5/071

(52) U.S. Cl. CPC C12N 5/0691; C12N 2513/00; C12N 2533/30; C12N 2533/50; C12N 2533/70; C12N 2539/00

(57) ABSTRACT

The present disclosure provides compositions, systems, and methods related to engineered vascular tissue models. In particular, the present disclosure provides compositions, systems, and methods pertaining to three-dimensional engineered vascular tissue models generated using vascular smooth muscle cells which emulate the structure and functionality of human vasculature.

### CROSS REFERENCE TO RELATED APPLICATIONS

This application claims priority to and the benefit of U.S. Provisional Patent Application No. 63/414,728 filed Oct. 10, 2023, which is incorporated herein by reference in its entirety and for all purposes.

### GOVERNMENT SUPPORT

This invention was made with Government support under Federal Grant no. RAD0102 awarded by the NASA Translational Research Institute for Space Health and NSF Graduate Research Fellowship Program (GRFP). The Federal Government has certain rights to this invention.

### FIELD

The present disclosure provides compositions, systems, and methods related to engineered vascular tissue models. In particular, the present disclosure provides compositions, systems, and methods pertaining to three-dimensional engineered vascular tissue models generated using vascular smooth muscle cells which emulate the structure and functionality of human vasculature.

### BACKGROUND

Cardiovascular disease (CVD) is the leading cause of mortality globally and within this group, there are a myriad of vascular diseases that when combined, make up a significant proportion of this burden. In this era of modern medicine, with access to technologies that can assess whole genomes, transcriptomes, biomes, and proteomes to accelerate biological discovery, the development and use of specific disease models is an innovative and increasingly powerful tool to study vascular diseases. This approach not only addresses the elusive clinical challenges for these diseases but also allows for in-depth investigation of disease-causing molecular mechanisms and phenotypes shared with vascular disease. Thus, generation of vascular models holds strong promise in treating patients with CVD and wound healing complications.

In one example, chronic exposure to ionizing radiation has been shown to damage the cardiovascular system significantly, putting various human populations at risk, particularly cancer survivors who have undergone radiotherapy and astronauts who will go on deep space missions. Many of the models used to study this phenomenon do not accurately re-capitulate to mechanisms of action by which this damage occurs in the human body. For example, animal models do not match humans on a genetic level, while many in vitro models use human cells but focus solely on the response of endothelial cells, and they usually do not facilitate native geometry or mechanical stimuli that impact cellular behavior.

### SUMMARY

Embodiments of the present disclosure include an engineered three-dimensional vascular tissue model. In accordance with these embodiments, the tissue model includes a biodegradable scaffold comprising a tubular structure and a plurality of vascular cells. As described further herein, the plurality of vascular cells is configured to surround the outer surface of the scaffold, thereby producing a tissue model that emulates the structure and functionality of human vasculature.

In some embodiments, the scaffold is comprised of natural materials. In other embodiments, the scaffold is comprised of synthetic materials.

In some embodiments, the scaffold comprises a polymer. In some embodiments, the polymer comprises at least one of polylactic acid (PLA), polyglycolic acid (PGA), polycaprolacttone (PCL), poly(D,L-lactide-co-glycolide) (PLGA), MPEG-PLGA (methoxypolyethyleneglycol)-poly(D,L-lactide-co-glycolide), polyhydroxyacids, and any combinations or derivatives thereof.

In some embodiments, the scaffold comprises at least one of gelatin, hyaluronan, hyaluronic acid (HA), chondroitin sulphate, dermatan sulphate, collagen, alginate, chitin, chitosan, keratin, silk, elastin, cellulose, ECM powder, thrombin, heparin sulfate, heparan sulfate, growth factors, fibrin, fibronectin, fibrinogen, tubulin, and any combinations or derivatives thereof.

In some embodiments, the scaffold is a single layer. In other embodiments, the scaffold is multilayered.

In some embodiments, the scaffold comprises at least one additional component selected from vascular endothelial growth factors (VEGFs), Fibroblast growth factor (FGFs), pleiotrophin (PTN), PRP (platelet rich plasma), Insulin-like growth factors (IGFs), Transforming growth factors (TGFs), Platelet-derived growth factors (PDGFs), Nerve growth factor (NGF), Human growth hormone (hGH), and Mechano growth factor (MGF).

In some embodiments, the scaffold comprises an elasticity modulus ranging from about 50 kPa to about 200 kPa. In some embodiments, the scaffold comprises a tensile strength ranging from about 50 kPa to about 500 kPa. In some embodiments, the scaffold comprises an internal diameter from about 0.4 mm to about 0.8 mm. In some embodiments, the model comprises a total wall thickness from about 0.4 mm to about 0.6 mm. In some embodiments, the scaffold comprises electrospun nanofibers.

In some embodiments, the plurality of vascular cells comprises at least one of endothelial cells, vascular smooth muscle cells, pericytes, fibroblasts, stem cells, and any combinations thereof. In some embodiments, the plurality of vascular cells comprise vascular smooth muscle cells. In some embodiments, the vascular smooth muscle cells are derived from human induced pluripotent stem cells. In some embodiments, the plurality of vascular cells do not comprise endothelial cells.

In some embodiments, the scaffold degrades after about 3 weeks after initial contact with the plurality of vascular cells.

Embodiments of the present disclosure also include a method of engineering a three-dimensional vascular tissue model. In accordance with these embodiments, the method includes obtaining a plurality of vascular cells, and applying the plurality of vascular cells to an outer surface of a biodegradable scaffold comprising a tubular structure to form a cellularized scaffold.

In some embodiments of the method, the plurality of vascular cells comprises at least one of endothelial cells, vascular smooth muscle cells, pericytes, fibroblasts, stem cells, and any combinations thereof. In some embodiments of the method, the plurality of vascular cells comprises vascular smooth muscle cells. In some embodiments of the method, the vascular smooth muscle cells are derived from human induced pluripotent stem cells. In some embodiments of the method, the plurality of vascular cells is engineered vascular cells. In some embodiments of the method, the plurality of vascular cells does not comprise endothelial cells.

In some embodiments of the method, the scaffold comprises a polymer. In some embodiments of the method, the polymer comprises at least one of polylactic acid (PLA), polyglycolic acid (PGA), polycaprolacttone (PCL), poly(D,L-lactide-co-glycolide) (PLGA), MPEG-PLGA (methoxypolyethyleneglycol)-poly(D,L-lactide-co-glycolide), polyhydroxyacids, and any combinations or derivatives thereof.

In some embodiments of the method, the polymer comprises at least one of gelatin, hyaluronan, hyaluronic acid (HA), chondroitin sulphate, dermatan sulphate, collagen, alginate, chitin, chitosan, keratin, silk, elastin, cellulose, ECM powder, thrombin, heparin sulfate, heparan sulfate, growth factors, fibrin, fibronectin, tubulin, fibrinogen, and any combinations or derivatives thereof.

In some embodiments of the method, the scaffold is generated using an electrospinning process.

In some embodiments of the method, the method further comprises attaching the cellularized scaffold to a bioreactor system. In some embodiments of the method, the bioreactor system is configured to apply pulsatile flow through the lumen of the cellularized scaffold.

In some embodiments of the method, the plurality of vascular cells are cultured in a two-dimensional culture plate prior to being applied to the biodegradable scaffold.

In some embodiments, the method further comprises allowing the cellularized scaffold to degrade, thereby resulting in a three-dimensional vascular tissue model.

Other aspects and embodiments of the disclosure will be apparent in light of the following detailed description and accompanying figures.

### BRIEF DESCRIPTION OF THE DRAWINGS

FIGS. 1A-1C: Ionizing radiation causes loss of cell adherence and dysregulation in extracellular matrix expression. (A) Cell count at 4 days after irradiation (n=5). (B) Confocal images of iSMCs stained for DAPI (blue), collagen I (white, left) and elastin (red, right) at 8 days after irradiation (scale bar=100 um). (C) Quantified collagen I intensity normalized to DAPI at 8 days after irradiation (left, n=2), quantified ratio of collagen I intensity to elastin intensity at 24 hours and 8 days after irradiation (right, n=1). Error bars represent SEM.

FIGS. 2A-2B: Smooth muscle tissue significantly increases total wall thickness of tissue-engineered small arteries. (A) Cross-sectional inner diameter and total wall thickness in fibrin only (black) and cellularized (pink) constructs measured via light microscopy show slightly decreased inner diameter and significantly increased wall thickness in constructs (**** indicates p<0.0001). (B) Representative images used for quantification show thickness and integration of added smooth muscle tissue in TESAs, resulting in a cohesive cellularized tissue-engineered construct.

FIG. 3: Fibrin outer wall thickness begins to degrade over 2 weeks after contact with iSMCs. Decreased fibrin graft wall thickness (left) and outer diameter (middle) but less pronounced change in inner diameter (right) indicates that the outer surface of the fibrin scaffolds, in contact with iSMCs, may begin to degrade over a period of two weeks in culture with iSMCs under pulsatile flow.

FIGS. 4A-4B: Vasodilation behavior is altered by iSMCs under pulsatile flow. (A) Diameter waveform for constructs subjected to 0.5 mL/min pulsatile flow (12 beats per minute) is altered in iSMC-seeded constructs. (B) Percent circumferential strain in outer diameter under increasing rates of pulsatile flow in fibrin only (left) or cellularized (right) constructs is tunable depending on fibrin thickness and increases to biologically relevant value of 2.5% using the thinnest fibrin scaffold construction.

FIGS. 5A-5C: Elasticity of iSMC-seeded constructs is increased compared to fibrin only grafts. (A) Normal force applied at ultimate construct failure during circumferential tensile testing was significantly increased in cellularized TESAs compared to fibrin only (* indicates p<0.05). (B) The lower slope for cellularized TESAs in plotted stress vs. strain curves and (C) significantly lower incremental elastic modulus in the elastic range of the stress test indicate that the iSMCs contribute to a significant increase in elasticity in cellularized TESAs vs. fibrin only scaffolds.

FIGS. 6A-6C: Smooth muscle tissue is multilayered, aligned, and cells express contractile markers. (A) Confocal imaging of immunostained iSMC-seeded constructs in low magnification en face orientation with both alpha smooth muscle actin (aSMA, red) and smooth muscle myosin heavy chain (SMMHC, green) show dense cell coverage over entire construct length. (B) En face high magnification imaging of external construct surface shows aligned contractile SMMHC fibers. (C) Images of TESA cross sections show significant tissue thickness and dense muscle cells with robust SMMHC expression.

FIGS. 7A-7C: iSMC-seeded constructs express anatomically accurate, aligned collagen fibers, and increased elastin expression compared to the same cells in 2D culture. Confocal imaging of iSMC-seeded constructs immunostained for Collagen Type I in either (A) en face orientation or (B) cross-sectional orientation shows circumferentially aligned and multilayered laminated mature collagen type I fibers. (C) Quantification of elastin production relative to cell count in 2D iSMCs and tissue engineered constructs, measured with the Fastin Elastin colorimetric protein quantitation assay shows increased elastin production in TESAs.

### DETAILED DESCRIPTION

Many of the currently available vascular models used to study the basic mechanisms of cardiovascular biology as well as relevant disease states and conditions do not accurately re-capitulate to mechanisms of action by which this damage occurs in the human body. For example, animal models do not match humans on a genetic level, while many in vitro models use human cells but focus solely on the response of endothelial cells, and they usually do not facilitate native geometry or mechanical stimuli that impact cellular behavior. By contrast, the various embodiments of the present disclosure include 3D arterial models, which uses tubular electrospun fibrin scaffolds cultured with human stem cell-derived vascular smooth muscle cells (iSMCs) in a bioreactor, in order to emulate the structure and functionality in the human vasculature, and to directly study changes to SMCs, including structural changes in the vasculature (e.g., extracellular matrix (ECM) remodeling). Thus, embodiments of the present disclosure include compositions, systems, and methods related to engineered vascular tissue models. In particular, the present disclosure provides compositions, systems, and methods pertaining to three-dimensional engineered vascular tissue models generated using vascular smooth muscle cells which emulate the structure and functionality of human vasculature.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

A cell has been “genetically modified,” “transduced,” “transformed,” or “transfected” by exogenous DNA, e.g., a recombinant expression vector, when such DNA has been introduced inside the cell. The presence of exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

“Optional” or “optionally” means that the subsequently described event or circumstance may or may not occur, and that the description includes instances where said event or circumstance occurs and instances where it does not.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician (e.g., physician).

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

Certain methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Engineered Tissue Models

Embodiments of the present disclosure include an engineered three-dimensional vascular tissue model. In accordance with these embodiments, the tissue model includes a biodegradable scaffold comprising a tubular structure and a plurality of vascular cells. As described further herein, the plurality of vascular cells are configured to surround the outer surface of the scaffold, thereby producing a tissue model that emulates the structure and functionality of human vasculature.

In some embodiments, the scaffold is comprised of natural materials. In some embodiments, the scaffold comprises at least one of gelatin, hyaluronan, hyaluronic acid (HA), chondroitin sulphate, dermatan sulphate, collagen, alginate, chitin, chitosan, keratin, silk, elastin, cellulose, ECM powder, thrombin, heparin sulfate, heparan sulfate, growth factors, fibrin, fibronectin, tubulin, fibrinogen, and any combinations or derivatives thereof. In some embodiments, the scaffold is comprised of synthetic materials. In some embodiments, the scaffold comprises a polymer. In some embodiments, the polymer comprises at least one of polylactic acid (PLA), polyglycolic acid (PGA), polycaprolacttone (PCL), poly(D,L-lactide-co-glycolide) (PLGA), MPEG-PLGA (methoxypolyethyleneglycol)-poly(D,L-lactide-co-glycolide), polyhydroxyacids, and any combinations or derivatives thereof. As would be recognized by one of ordinary skill in the art based on the present disclosure, various combinations of these and other materials can be used to generate the scaffolds described herein, and the materials listed above are not intended to be limiting. In some embodiments, the scaffold is comprised of a single layer. In other embodiments, the scaffold is comprised of more than one layer (e.g., multilayered). As described further herein, one exemplary embodiment of the scaffold comprises electrospun nanofibers.

In accordance with these embodiments, the engineered vascular tissue models of the present disclosure can be generated to include various other components. In some embodiments, these additional components can facilitate cell and/or tissue growth within and around the scaffold. In some embodiments, these additional components can provide biophysical or biomechanical properties to facilitate the binding and/or growth of the cells and/or tissues on the scaffold. Therefore, in some embodiments, the scaffold can be engineered to include at least one additional component, including but not limited to, vascular endothelial growth factors (VEGFs), Fibroblast growth factor (FGFs), pleiotrophin (PTN), PRP (platelet rich plasma), Insulin-like growth factors (IGFs), Transforming growth factors (TGFs), Platelet-derived growth factors (PDGFs), Nerve growth factor (NGF), Human growth hormone (hGH), and Mechano growth factor (MGF).

In some embodiments, the engineered vascular tissue models of the present disclosure can be generated such that the scaffold comprises a certain elasticity modulus depending on the materials used to generate the scaffold and based on a certain applied strain. In some embodiments, the scaffold comprises an elasticity ranging from about 50 kPa to about 250 kPa. In some embodiments, the scaffold comprises an elasticity ranging from about 50 kPa to about 200 kPa. In some embodiments, the scaffold comprises an elasticity ranging from about 50 kPa to about 150 kPa. In some embodiments, the scaffold comprises an elasticity ranging from about 50 kPa to about 100 kPa. In some embodiments, the scaffold comprises an elasticity ranging from about 100 kPa to about 250 kPa. In some embodiments, the scaffold comprises an elasticity ranging from about 150 kPa to about 250 kPa. In some embodiments, the scaffold comprises an elasticity ranging from about 200 kPa to about 250 kPa. In some embodiments, the scaffold comprises an elasticity ranging from about 100 kPa to about 200 kPa. In some embodiments, the scaffold comprises an elasticity ranging from about 150 kPa to about 200 kPa. In some embodiments, the scaffold comprises an elasticity ranging from about 100 kPa to about 150 kPa.

In some embodiments, the engineered vascular tissue models of the present disclosure can be generated such that the scaffold comprises a tensile strength that is adjustable for different applications (e.g., modeling different aspects or types of vasculature). In some embodiments, the scaffold comprises a tensile strength ranging from about 50 kPa to about 500 kPa. In some embodiments, the scaffold comprises a tensile strength ranging from about 50 kPa to about 450 kPa. In some embodiments, the scaffold comprises a tensile strength ranging from about 50 kPa to about 400 kPa. In some embodiments, the scaffold comprises a tensile strength ranging from about 50 kPa to about 350 kPa. In some embodiments, the scaffold comprises a tensile strength ranging from about 50 kPa to about 300 kPa. In some embodiments, the scaffold comprises a tensile strength ranging from about 50 kPa to about 250 kPa. In some embodiments, the scaffold comprises a tensile strength ranging from about 50 kPa to about 200 kPa. In some embodiments, the scaffold comprises a tensile strength ranging from about 50 kPa to about 150 kPa. In some embodiments, the scaffold comprises a tensile strength ranging from about 50 kPa to about 100 kPa. In some embodiments, the scaffold comprises a tensile strength ranging from about 100 kPa to about 500 kPa. In some embodiments, the scaffold comprises a tensile strength ranging from about 150 kPa to about 500 kPa. In some embodiments, the scaffold comprises a tensile strength ranging from about 200 kPa to about 500 kPa. In some embodiments, the scaffold comprises a tensile strength ranging from about 250 kPa to about 500 kPa. In some embodiments, the scaffold comprises a tensile strength ranging from about 300 kPa to about 500 kPa. In some embodiments, the scaffold comprises a tensile strength ranging from about 350 kPa to about 500 kPa. In some embodiments, the scaffold comprises a tensile strength ranging from about 400 kPa to about 500 kPa. In some embodiments, the scaffold comprises a tensile strength ranging from about 450 kPa to about 500 kPa. In some embodiments, the scaffold comprises a tensile strength ranging from about 100 kPa to about 400 kPa. In some embodiments, the scaffold comprises a tensile strength ranging from about 150 kPa to about 350 kPa. In some embodiments, the scaffold comprises a tensile strength ranging from about 200 kPa to about 400 kPa. In some embodiments, the scaffold comprises a tensile strength ranging from about 250 kPa to about 350 kPa.

In some embodiments, the engineered vascular tissue models of the present disclosure can be generated such that the scaffold comprises a thickness that is adjustable for different applications (e.g., modeling different aspects or types of vasculature). In general, the thickness of the vascular model will depend on the corresponding thickness of the scaffold and the layer of vascular cells. In some embodiments, the thickness of the scaffold ranges from about 0.1 mm to about 0.3 mm. In some embodiments, the thickness of the vascular cells (e.g., cellularized vasculature (e.g., with iSMCs)) layered on the scaffold ranges from about 0.3 mm to about 0.5 mm. In accordance with these embodiments, the wall thickness of the engineered vascular tissue model can range from about 0.4 mm to about 0.8 mm.

In some embodiments, the engineered vascular tissue models of the present disclosure can be generated such that the scaffold comprises an internal diameter that is adjustable for different applications (e.g., modeling different aspects or types of vasculature). In some embodiments, the scaffold comprises an internal diameter from about 0.2 mm to about 1.0 mm. In some embodiments, the scaffold comprises an internal diameter from about 0.2 mm to about 0.9 mm. In some embodiments, the scaffold comprises an internal diameter from about 0.2 mm to about 0.8 mm. In some embodiments, the scaffold comprises an internal diameter from about 0.2 mm to about 0.7 mm. In some embodiments, the scaffold comprises an internal diameter from about 0.2 mm to about 0.6 mm. In some embodiments, the scaffold comprises an internal diameter from about 0.2 mm to about 0.5 mm. In some embodiments, the scaffold comprises an internal diameter from about 0.2 mm to about 0.4 mm. In some embodiments, the scaffold comprises an internal diameter from about 0.2 mm to about 0.3 mm. In some embodiments, the scaffold comprises an internal diameter from about 0.3 mm to about 1.0 mm. In some embodiments, the scaffold comprises an internal diameter from about 0.4 mm to about 1.0 mm. In some embodiments, the scaffold comprises an internal diameter from about 0.5 mm to about 1.0 mm. In some embodiments, the scaffold comprises an internal diameter from about 0.6 mm to about 1.0 mm. In some embodiments, the scaffold comprises an internal diameter from about 0.7 mm to about 1.0 mm. In some embodiments, the scaffold comprises an internal diameter from about 0.8 mm to about 1.0 mm. In some embodiments, the scaffold comprises an internal diameter from about 0.9 mm to about 1.0 mm. In some embodiments, the scaffold comprises an internal diameter from about 0.4 mm to about 0.8 mm. In some embodiments, the scaffold comprises an internal diameter from about 0.3 mm to about 0.7 mm. In some embodiments, the scaffold comprises an internal diameter from about 0.4 mm to about 0.6 mm.

As would be recognized by one of ordinary skill in the art based on the present disclosure, the engineered vascular tissue models described herein can be constructed to include one or more types of vascular cells. In some embodiments, the plurality of vascular cells comprises at least one of endothelial cells, vascular smooth muscle cells, pericytes, fibroblasts, stem cells, and any combinations thereof. In some embodiments, the plurality of vascular cells comprise vascular smooth muscle cells. In some embodiments, the vascular smooth muscle cells are derived from human induced pluripotent stem cells. In some embodiments, the plurality of vascular cells do not comprise endothelial cells.

As described further herein, the engineered vascular tissue models of the present disclosure can comprise a scaffold that is designed to degrade over a period of time, such that the vascular cells remain largely intact as a tubular structure. In some embodiments, the scaffold degrades within about 1 week after initial contact with the plurality of vascular cells. In some embodiments, the scaffold degrades within about 2 weeks after initial contact with the plurality of vascular cells. In some embodiments, the scaffold degrades within about 3 weeks after initial contact with the plurality of vascular cells. In some embodiments, the scaffold degrades within about 4 weeks after initial contact with the plurality of vascular cells. In some embodiments, the scaffold degrades within about 6 to 7 days after initial contact with the plurality of vascular cells. In some embodiments, the scaffold degrades within about 5 to 6 days after initial contact with the plurality of vascular cells. In some embodiments, the scaffold degrades within about 4 to 5 days after initial contact with the plurality of vascular cells. In some embodiments, the scaffold degrades within about 3 to 4 days after initial contact with the plurality of vascular cells. In some embodiments, the scaffold degrades within about 2 to 3 days after initial contact with the plurality of vascular cells. In some embodiments, the scaffold degrades within about 1 to 2 days after initial contact with the plurality of vascular cells.

Embodiments of the present disclosure also include a method of engineering any of the three-dimensional vascular tissue models described herein. In accordance with these embodiments, the method includes obtaining a plurality of vascular cells, and applying the plurality of vascular cells to an outer surface of a biodegradable scaffold comprising a tubular structure to form a cellularized scaffold. In some embodiments of the method, the plurality of vascular cells are cultured in a two-dimensional culture plate prior to being applied to the biodegradable scaffold.

As described herein, the plurality of vascular cells comprises at least one of endothelial cells, vascular smooth muscle cells, pericytes, fibroblasts, stem cells, and any combinations thereof. In some embodiments of the method, the plurality of vascular cells comprise vascular smooth muscle cells. In some embodiments of the method, the vascular smooth muscle cells are derived from human induced pluripotent stem cells. In some embodiments of the method, the plurality of vascular cells are engineered vascular cells. In some embodiments of the method, the plurality of vascular cells do not comprise endothelial cells.

In some embodiments of the method, and as described further herein, the scaffold comprises a polymer. In some embodiments of the method, the polymer comprises at least one of polylactic acid (PLA), polyglycolic acid (PGA), polycaprolacttone (PCL), poly(D,L-lactide-co-glycolide) (PLGA), MPEG-PLGA (methoxypolyethyleneglycol)-poly(D,L-lactide-co-glycolide), polyhydroxyacids, and any combinations or derivatives thereof. In some embodiments of the method, the polymer comprises at least one of gelatin, hyaluronan, hyaluronic acid (HA), chondroitin sulphate, dermatan sulphate, collagen, alginate, chitin, chitosan, keratin, silk, elastin, cellulose, ECM powder, thrombin, heparin sulfate, heparan sulfate, growth factors, fibrin, fibronectin, tubulin, fibrinogen, and any combinations or derivatives thereof.

In some embodiments, the method further comprises generating the scaffold using an electrospinning process. As would be recognized by one of ordinary skill in the art based on the present disclosure, electrospinning is a technique that uses polymer solutions and strong electric fields to produce nano-sized fibers that have wide-ranging applications, including the generation of scaffolds used in the three-dimensional vascular tissue models described herein.

In some embodiments, the method further comprises attaching the cellularized scaffold to a bioreactor system. In some embodiments of the method, the bioreactor system is configured to apply pulsatile flow through the lumen of the cellularized scaffold. In some embodiments, the method further comprises allowing the cellularized scaffold to degrade, thereby resulting in a three-dimensional vascular tissue model.

Other aspects and embodiments of the disclosure will be apparent in light of the following detailed description and accompanying figures.

### 3. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

The iSMCs are derived from human induced pluripotent stem cells using established protocols. For our 3D human vascular model, the iSMCs are removed from 2D culture plates as a single sheet and wrapped around a 0.6 mm ID tubular electrospun fibrin scaffold. The cellularized scaffold is sutured into a bioreactor system and cultured with pulsatile flow through the lumen. The bioreactors are then exposed to either 0.5 Gy or 10 Gy of X-ray irradiation at 160 KeV for optimization, and to 0.1, 0.25, or 0.5 Gy of simulated Galactic Cosmic Radiation (GCR) at the NASA Space Radiation Laboratory. Samples are immunostained with ethidiumhomodimer-1/calcein AM, Ki67, smooth muscle mysosin heavy chain (SM-MHC), collagen I, and elastin, then they are imaged and quantified.

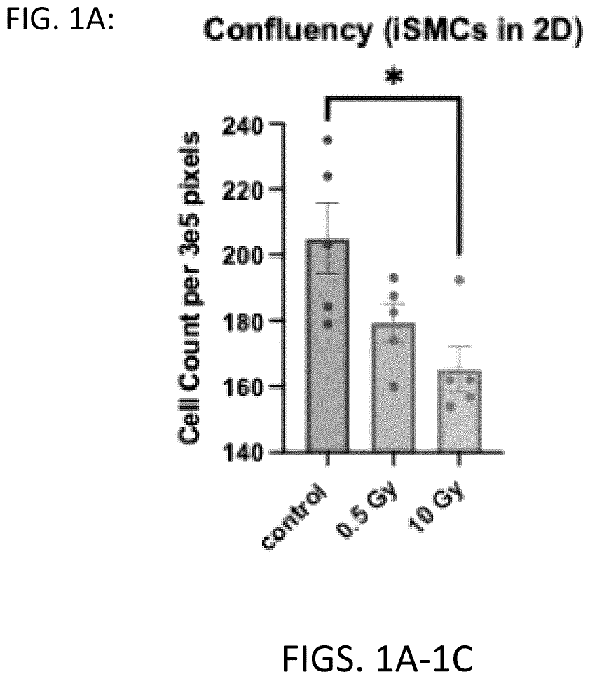

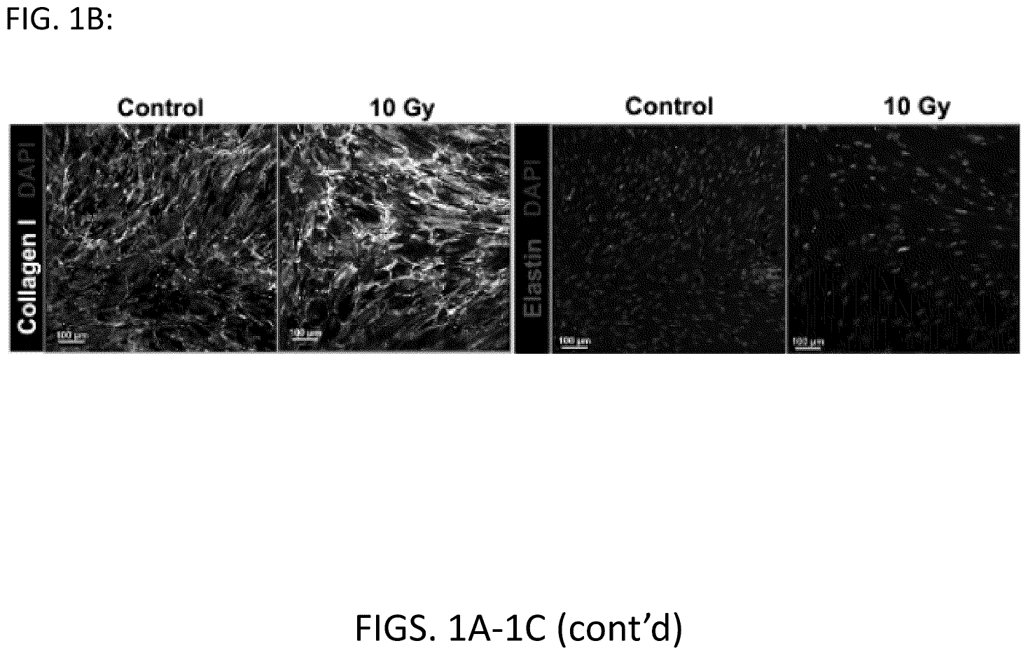

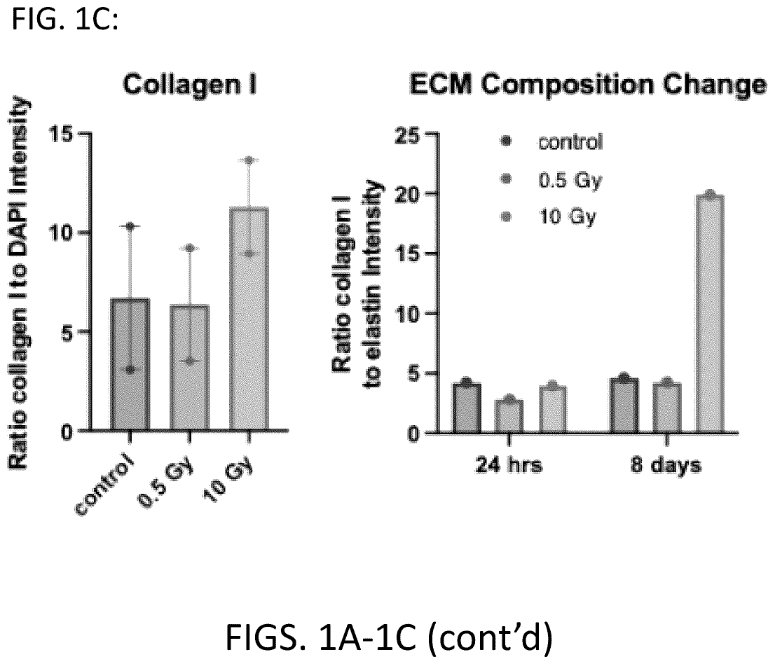

Irradiated iSMC samples show increased cell death, decreased proliferation rate, and decreased confluency in a dose-dependent manner after exposure, with significantly fewer cells present in 10 Gy samples compared to controls after 4 days (FIG. 1A). Interestingly, increased production of collagen I by iSMCs was seen at 8 days post-irradiation, while elastin production decreased slightly, indicating that ECM composition changes over time after exposure (FIGS. 1B-1C).

### Example 2

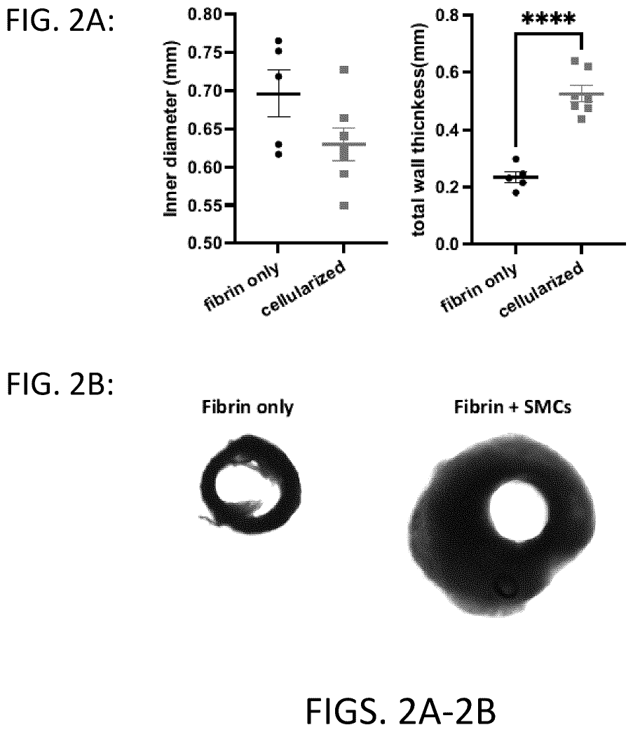

Previous handling. Smooth muscle tissue significantly increases total wall thickness of tissue engineered small artery (TESA) model. The iSMC-seeded tissue engineered small artery (TESA) constructs and fibrin only scaffolds are cultured in the pulsatile flow bioreactor system for 7 days at a constant flow rate of 0.5 mL/min and 12 beats per minute. Samples are excised from bioreactors, dissected into 0.5 mm long sections, and imaged via light microscopy while positioned upright (FIGS. 2A-2B). The average outer and inner diameter for fibrin only scaffold or TESAs in the resulting cross sectional images are measured with pixel to micron conversion.

### Example 3

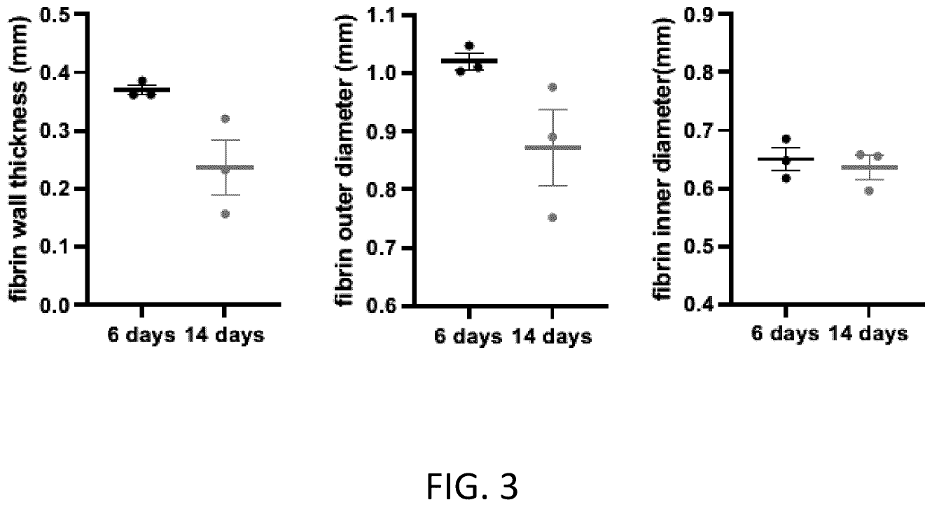

Circumferential strain (vasodilation) is altered by iSMCs under pulsatile flow. After iSMCs are seeded on the fibrin scaffolds, TESAs are cultured under constant pulsatile flow (same regime as above) for 6 days or 14 days. TESAs are excised from bioreactors, fixed in 3.7% formaldehyde for 1 hour, and frozen in Optimal Cutting Temperature (OCT) medium at −80 degrees Celsius. Frozen samples are then sectioned into 5 micron sections, attached to glass slides, and immunostained for proteins of interest. Using confocal fluorescent microscopy, the fibrin scaffold is clearly distinguished from the smooth muscle tissue due to fibrin autofluorescence emission, and average outer and inner fibrin diameter is measured with pixel to micron conversion (FIG. 3).

### Example 4

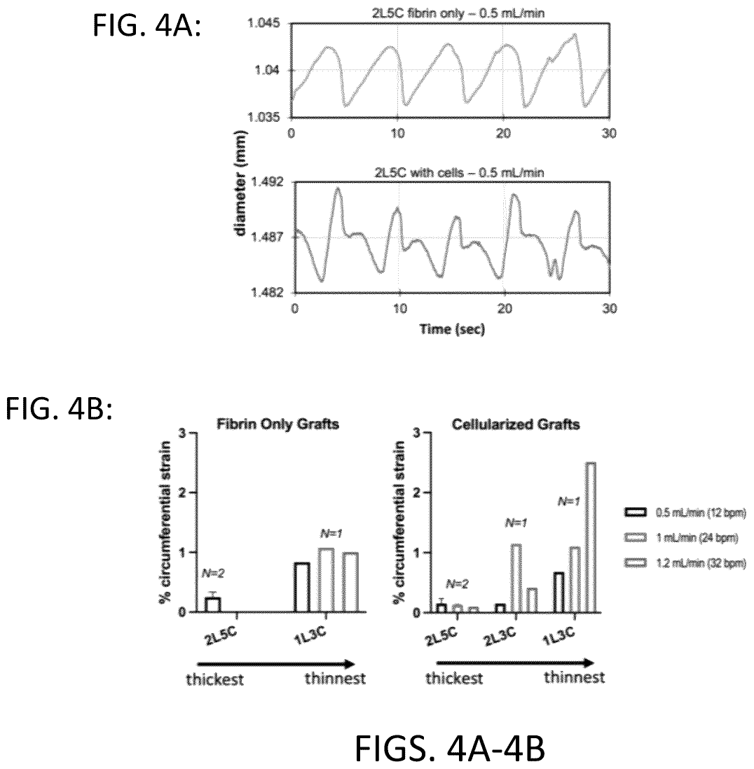

Elasticity of iSMC-seeded TEVGs is increased compared to scaffold only in fibrin grafts. After TESAs or fibrin-only scaffolds are cultured under constant pulsatile flow (same regime as example 2) for 7 days, media is exchanged for phenol red-free equivalent media and the bioreactor optical transparency is utilized to measure dynamic outer diameter changes of constructs with optical micrometry. Bioreactors cultured under three different pulsatile flow rates are placed under a laser calibrated to precisely measure diameter of optically opaque objects (the TESAs) in real-time. Diameter change in fibrin only grafts and TESAs is measured at 0.5 mL/min (12 beats/min), 1 mL/min (24 beats/min), and 1.2 mL/min (32 beats/min) for 2 minutes. Circumferential strain is calculated using the average and peak diameter measured over the 2 minute time period (FIGS. 4A-4B).

### Example 5

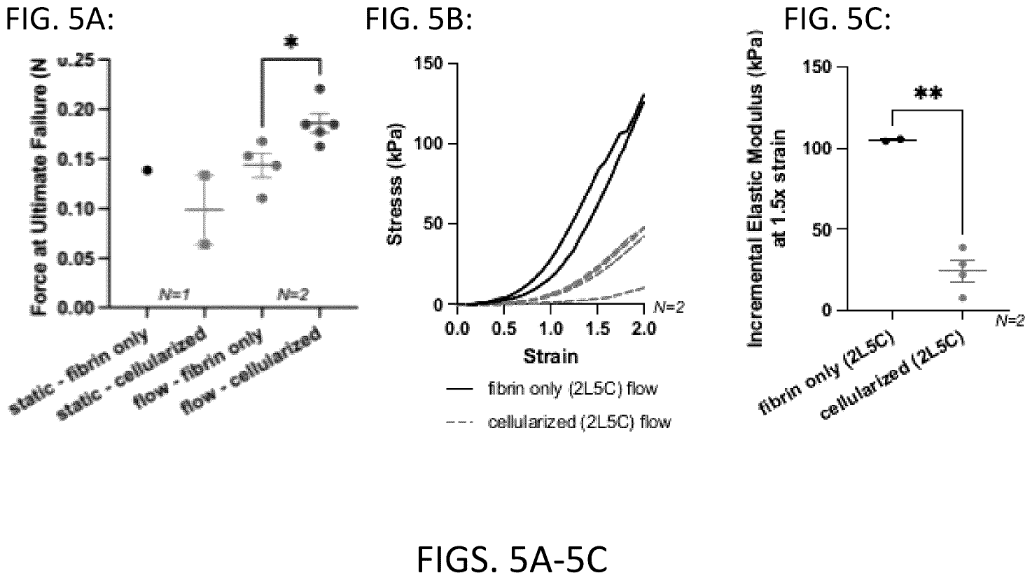

Smooth Muscle Tissue is multilayered, aligned, and cells express contractile markers. After culture in pulsatile flow (same regime as in example 2) for 7 days, samples were excised from bioreactors and dissected into 2 mm sections. The dimensions including wall thickness and exact section length were measured via light microscopy, and sections were then mounted circumferentially onto 2 parallel hooks which apply tensile force to pull the section radially at 50 microns/second until complete failure when the construct ripped entirely (FIG. 5A, * indicates p<0.05). Applied force vs. distance measurements were normalized to stress vs. strain using sample dimensions, and the resultant stress/strain curves were plotted (FIG. 5B). The incremental elastic modulus was calculated at the chosen strain value of 1.5, which is in the elastic range of deformation (FIG. 5C, ** indicates p<0.01).

### Example 6

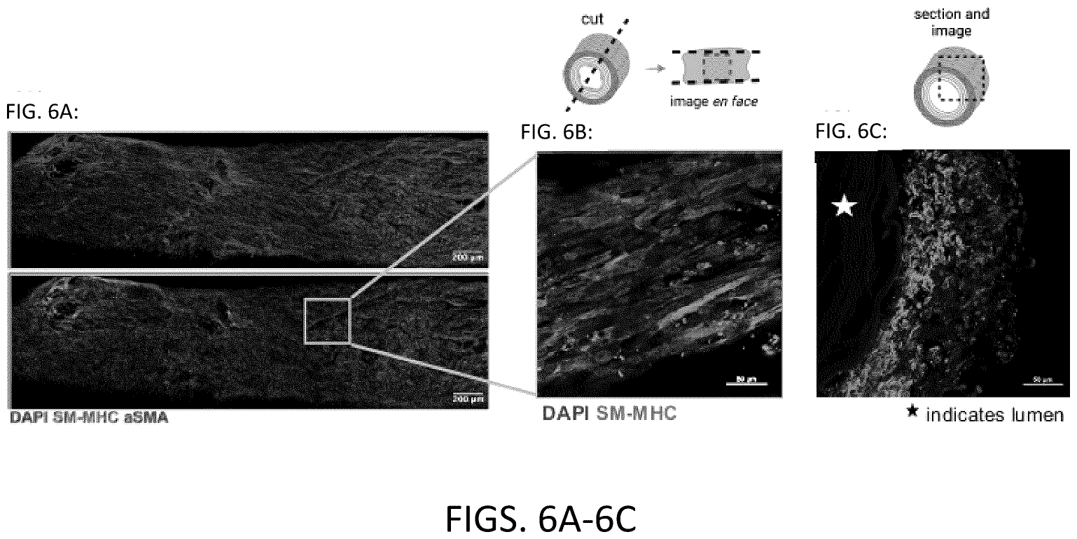

iSMC-seeded TEVGs express anatomically accurate, aligned collagen fibers, and increased elastin expression compared to the same cells in 2D. Cellularized TESAs cultured under pulsatile flow (same regime as Example 2) for at least 3 days were excised from bioreactors, fixed in 3.7% formaldehyde for 1 hour, blocked in 5% BSA, and immunostained with anti-human antibodies for contractile markers smooth muscle myosin heavy chain (SMMHC) and alpha smooth muscle actin (aSMA). Whole TESAs were mounted on slides and the entire external surface and all cellular tissue was imaged using fluorescence confocal microscopy (FIG. 6A). High magnification of the same sample was used to visualize cell morphology and contractile protein fibers (FIG. 6B). Simultaneously, after formaldehyde fixation, a section of each TESA was cut from the sample, mounted in OCT, frozen, and sectioned in 5 micron sections. These cross sections were then immunostained for contractile markers and imaged using high magnification to view tissue features in detail (FIG. 6C).

### Example 7

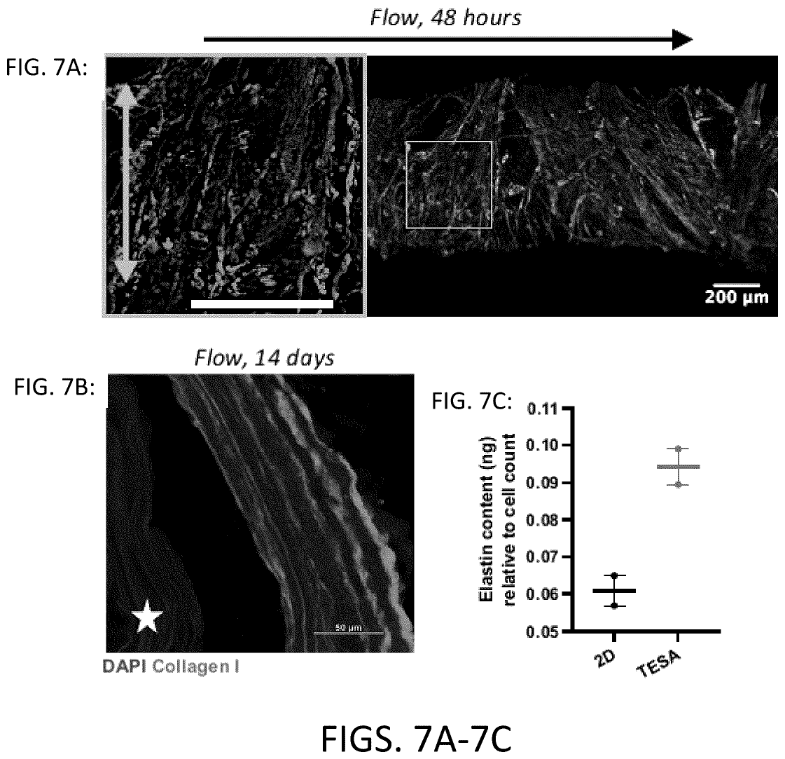

iSMC-seeded constructs express anatomically accurate, aligned collagen fibers, and increased elastin expression compared to the same cells in 2D culture. Cellularized TESAs were cultured under pulsatile flow (same regime as Example 2) for at least 48 hours (FIG. 7A) or for at least 14 days (FIG. 7B). Samples were excised from bioreactors, fixed in 3.7% formaldehyde, and immediately stained (or mounted in OCT, sectioned, and stained) using the same protocol as in Example 6, substituting the antibody for anti-human collagen type I. Whole mount or sectioned samples were then imaged using fluorescence confocal microscopy. To quantify mature elastin production, iSMCs cultured on collagen-coated plates for 8 days or TESAs cultured in bioreactors for 8 days were excised, minced, and added to the working reagents for the Fastin Elastin Colorimetric Assay (Biocolor Assay) following the manufacturers protocol. After completion of the assay protocol, the absorbance measured at 562 nm with a plate reader was used to calculate elastin concentration in each sample (FIG. 7C).

What is claimed is:

1. An engineered three-dimensional vascular tissue model, comprising:
a biodegradable scaffold comprising a tubular structure; and
a plurality of vascular cells;
wherein the plurality of vascular cells are configured to surround the outer surface of the scaffold.

2. The vascular tissue model of claim 1, wherein the scaffold is natural or synthetic.

3. The vascular tissue model of claim 1, wherein the scaffold comprises a polymer.

4. The vascular tissue model of claim 3, wherein the polymer comprises at least one of polylactic acid (PLA), polyglycolic acid (PGA), polycaprolacttone (PCL), poly(D,L-lactide-co-glycolide) (PLGA), MPEG-PLGA (methoxypolyethyleneglycol)-poly(D,L-lactide-co-glycolide), polyhydroxyacids, and any combinations or derivatives thereof.

5. The vascular tissue model of claim 1, wherein the scaffold comprises at least one of gelatin, hyaluronan, hyaluronic acid (HA), chondroitin sulphate, dermatan sulphate, collagen, alginate, chitin, chitosan, keratin, silk, elastin, cellulose, ECM powder, thrombin, heparin sulfate, heparan sulfate, growth factors, fibrin, fibronectin, tubulin, and any combinations or derivatives thereof.

6. The vascular tissue model of claim 1, wherein the scaffold comprises at least one additional component selected from vascular endothelial growth factors (VEGFs), Fibroblast growth factor (FGFs), pleiotrophin (PTN), PRP (platelet rich plasma), Insulin-like growth factors (IGFs), Transforming growth factors (TGFs), Platelet-derived growth factors (PDGFs), Nerve growth factor (NGF), Human growth hormone (hGH), and Mechano growth factor (MGF).

7. The vascular tissue model of claim 1, wherein the scaffold comprises an elasticity modulus ranging from about 50 kPa to about 200 kPa.

8. The vascular tissue model of claim 1, wherein:
(i) the scaffold comprises a tensile strength ranging from about 50 kPa to about 500 kPa; and/or
(ii) the scaffold comprises an internal diameter from about 0.4 mm to about 0.8 mm.

9. The vascular tissue model of claim 1, wherein the plurality of vascular cells comprises at least one of endothelial cells, vascular smooth muscle cells, pericytes, fibroblasts, stem cells, and any combinations thereof.

10. The vascular tissue model of claim 1, wherein the plurality of vascular cells comprise vascular smooth muscle cells.

11. The vascular tissue model of claim 10, wherein the vascular smooth muscle cells are derived from human induced pluripotent stem cells.

12. The vascular tissue model of claim 1, wherein the plurality of vascular cells do not comprise endothelial cells.

13. A method of engineering a three-dimensional vascular tissue model, the method comprising:
obtaining a plurality of vascular cells; and
applying the plurality of vascular cells to an outer surface of a biodegradable scaffold comprising a tubular structure to form a cellularized scaffold.

14. The method of claim 13, wherein the plurality of vascular cells comprises at least one of endothelial cells, vascular smooth muscle cells, pericytes, fibroblasts, stem cells, and any combinations thereof.

15. The method of claim 13, wherein the plurality of vascular cells comprise vascular smooth muscle cells.

16. The method of claim 15, wherein the vascular smooth muscle cells are derived from human induced pluripotent stem cells.

17. The method of claim 13, wherein the plurality of vascular cells do not comprise endothelial cells.

18. The method of claim 13, wherein the scaffold comprises a polymer.

19. The method of claim 13, wherein the polymer comprises at least one of polylactic acid (PLA), polyglycolic acid (PGA), polycaprolacttone (PCL), poly(D,L-lactide-co-glycolide) (PLGA), MPEG-PLGA (methoxypolyethyleneglycol)-poly(D,L-lactide-co-glycolide), polyhydroxyacids, and any combinations or derivatives thereof.

20. The method of claim 13, wherein the polymer comprises at least one of gelatin, hyaluronan, hyaluronic acid (HA), chondroitin sulphate, dermatan sulphate, collagen, alginate, chitin, chitosan, keratin, silk, elastin, cellulose, ECM powder, thrombin, heparin sulfate, heparan sulfate, growth factors, fibrin, fibronectin, tubulin, and any combinations or derivatives thereof.
